(19) United States
(12) Patent Application Publication
(10) Pub. No.: US 2023/0109998 A1
(43) Pub. Date: Apr. 13, 2023

(54) MALARIA TRANSMISSION-BLOCKING VACCINES

(71) Applicants: PATH, Seattle, WA (US); National University Corporation Ehime University, Matsuyama-shi, Ehime (JP); Sumitomo Pharma Co., Ltd., Osaka-shi, Osaka (JP)

(72) Inventors: C. Richter King, Washington, DC (US); Yimin Wu, Germantown, MD (US); Jordan Lee Plieskatt, Alexandria, VA (US); Shwu-Maan Lee, Rockville, MD (US); Chia-Kuel Wu, Rockville, MD (US); Takafumi Tsuboi, Matsuyama-shi, Ehime (JP); Akihisa Fukushima, Osaka-shi, Osaka (JP)

(21) Appl. No.: 17/913,699

(22) PCT Filed: Nov. 6, 2020
(86) PCT No.: PCT/JP2020/041583

§ 371 Date: Sep. 22, 2022

Related U.S. Application Data
(60) Provisional application No. 63/000,639, filed on Mar. 27, 2020.

(51) Int. Cl. A61K 39/39; A61K 39/015; A61K 31/505; A61P 33/06

(52) U.S. Cl. CPC A61K 39/39; A61K 39/015; A61K 31/505; A61P 33/06; A61K 2039/55511

(57) ABSTRACT

Malaria transmission-blocking vaccines with good preservation stability and immunostimulatory action are provided. According the present invention, combination use of a pharmaceutical composition comprising (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide, or a pharmaceutically acceptable salt thereof, as a vaccine adjuvant with enhanced specific immune response against antigens and good preservation stability and a malaria vaccine with non-glycosylation, homogeneity, and biological activity allow for the provision of malaria transmission-blocking vaccines with good preservation stability and immunostimulatory action.

### TECHNICAL FIELD

The present invention relates to combination use of a pharmaceutical composition comprising a compound, or a pharmaceutically acceptable salt thereof, useful for a vaccine adjuvant and a malaria vaccine, and a method for blocking transmission of malaria parasites.

### BACKGROUND ART

Sub-unit vaccines where a part of components of a pathogen is used for an antigen can be prepared by chemical synthesis and genetic recombination, and such sub-unit vaccines are more useful than vaccines prepared from a pathogen itself in terms of safety and preparation methods of vaccines. Sub-unit vaccines, however, tend to show lower immunostimulatory action than live vaccines and inactivated vaccines prepared from a pathogen itself do. In order to enhance immunogenicity of epitopes and improve immunostimulatory action of vaccines, combination use of a vaccine antigen and an adjuvant has been studied for prevention and treatment for diseases.

Recently, (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaenamide, referred to as “Compound A” hereinafter, as shown below has been reported as an adjuvant having TLR7 agonistic activity (PTL 1).

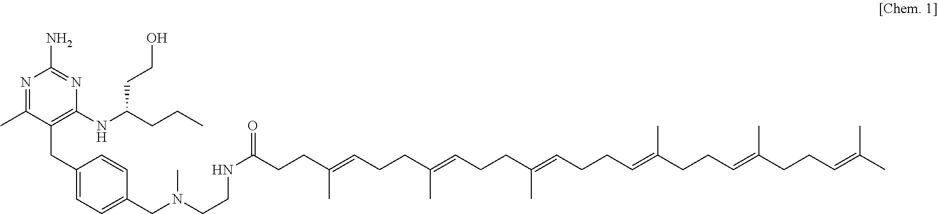 [SMILES: CCC[C@@H](CCO)Nc1nc(N)nc(C)c1Cc1ccc(CN(C)CCNC(=O)CC/C(C)=C/CC/C(C)=C/CC/C(C)=C/CC/C=C(\C)CC/C=C(\C)CCC=C(C)C)cc1]

Compound A has the good vaccine-adjuvant activity, but it is required to be formulated in a formulation such as emulsions when administered to mammals as a vaccine adjuvant. In general, it is known that emulsion formulations comprise antioxidant agents such as ascorbic acids to improve the preservation stability in formulations. It has, however, not been known that antioxidant agents such as ascorbic acids can stabilize particle-size distribution.

It has been known that a gametocyte surface protein of malaria parasites, Plasmodium falciparum, Pfs230, consisting of 3,135 amino acids is a target antigen for malaria transmission-blocking vaccine. Due to the large size, complex domains, and repeating seven-cysteine (7-Cys) motifs with a multitude of disulfide bonds (NPL 3), the feasibility of expression of a full-length protein has been difficult. A focus, therefore, has been on the generation of single domains, including N-terminal fragments. Among such fragments, some Pfs230 fragments including Pfs230C1 and Pfs230D1 have been reported (NPLs 1 and 2). Some of such proteins, however, have been known to cause glycosylation, and improved antigens with non-glycosylation, homogeneity, and biological activity have been desired.

A known malaria vaccine acquires immunogenicity enhanced by an adjuvant, Alhydrogel (R) (NPL 2). Alhydrogel is different from Compound A.

### CITATION LIST

### Patent Literature

[PTL 1] WO 2017/061532

### Non Patent Literature

[NPL 1] Shwu-Maan Lee et al., Clinical and Vaccine Immunology 2017; 24:e00140
[NPL 2] Camila H. Coelho et al., Vaccine 37 (2019) 1038-1045
[NPL 3] Mayumi Tachibana et al., Clinical and Vaccine Immunology v. 18(8); 2011, 1343-1350

### SUMMARY OF INVENTION

### Technical Problem

The present invention provides combination use of a pharmaceutical composition comprising a compound, or a pharmaceutically acceptable salt thereof, useful for a vaccine adjuvant with good preservation stability and immunostimulatory action and a malaria transmission-blocking vaccine, and a method for blocking transmission of malaria parasites comprising administering the pharmaceutical composition and the malaria transmission-blocking vaccine to mammals.

### Solution to Problem

Compound A has six unsaturated bonds derived from the intramolecular squalene-like structure. During studies of formulations of Compound A, it has been found that when Compound A is formulated into lyophilized formulations of common emulsion formulations with squalene as an oil composition, the intramolecular unsaturated bonds are oxidized, and thereby, the content of Compound A decreases. After further studies by the inventors to provide formulations of vaccine adjuvants with feasible preservation stability, improved stability against oxidation of Compound A has been achieved by using squalane as an oil composition in formulation of an emulsion composition of Compound A. In addition, formulations with good preservation stability, particularly the stability of particle-size distributions as well as the oxidative stability of Compound A itself have been achieved by addition of an antioxidant agent such as ascorbic acids.

The inventors have also found that an improved N-terminal Pfs230 fragment as a malaria transmission-blocking vaccine is non-glycosylated, homogeneous, and biologically active.

The inventors have also found that combination use of a pharmaceutical composition comprising Compound A and a malaria vaccine comprising an improved N-terminal Pfs230 fragment is useful for malaria transmission-blocking vaccines with good preservation stability and immunostimulatory action.

Embodiments of the present invention are illustrated as follows.

Item 1. A method for blocking transmission of malaria parasites from a human to a mosquito, comprising administering a pharmaceutically effective amount of a combination of I) a pharmaceutical composition and II) a vaccine to a human residing in an area in need of blocking malaria transmission, wherein:
I) the pharmaceutical composition comprises the following ingredients i) to vi):

i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide, referred to as “Compound A” hereinafter, or a pharmaceutically acceptable salt thereof;

ii) squalane;

iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;

iv) an excipient selected from the group consisting of non-reducing sugars and sugar alcohols, except for mannitol;

v) a hydrophilic surfactant; and

vi) a lipophilic surfactant; and

II) the vaccine is a malaria vaccine comprising an antigen having the sequence represented by SEQ ID NO: 1 or SEQ ID NO: 2.
Item 2. A combination drug comprising:
I) a pharmaceutical composition comprising:

i) (4E,8E,12E,16E,20E)-N-{2-[{4-(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;

ii) squalane;

iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;

iv) an excipient selected from the group consisting of non-reducing sugars and sugar alcohols, except for mannitol;

v) a hydrophilic surfactant; and

vi) a lipophilic surfactant; and

II) a malaria vaccine comprising an antigen having the sequence represented by SEQ ID NO: 1 or SEQ ID NO: 2.
Item 3. A vaccine formulation for malaria comprising:
I) a pharmaceutical composition comprising:

i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;

ii) squalane;

iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;

iv) an excipient selected from the group consisting of non-reducing sugars and sugar alcohols, except for mannitol;

v) a hydrophilic surfactant; and

vi) a lipophilic surfactant; and

II) a malaria vaccine comprising an antigen having the sequence represented by SEQ ID NO: 1 or SEQ ID NO: 2.
Item 4. A kit comprising:
I) a pharmaceutical composition comprising:

i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;

ii) squalane;

iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;

iv) an excipient selected from the group consisting of non-reducing sugars and sugar alcohols, except for mannitol;

v) a hydrophilic surfactant; and

vi) a lipophilic surfactant; and

II) a malaria vaccine comprising an antigen having the sequence represented by SEQ ID NO: 1 or SEQ ID NO: 2.
Item 5. The method, the combination drug, the vaccine formulation, or the kit according to any one of Items 1 to 4, wherein the pharmaceutical composition is an oil-in-water type emulsion formulation or a lyophilized formulation thereof.
Item 6. The method, the combination drug, the vaccine formulation, or the kit according to any one of Items 1 to 5, wherein the hydrophilic surfactant is polyoxyethylene sorbitan fatty acid esters (e.g., polysorbate 20, polysorbate 40, polysorbate 60, polysorbate 65, and polysorbate 80); polyoxyethylene hydrogenated castor oils (e.g., polyoxyethylene hydrogenated castor oil 10, polyoxyethylene hydrogenated castor oil 20, polyoxyethylene hydrogenated castor oil 40, polyoxyethylene hydrogenated castor oil 50, and polyoxyethylene hydrogenated castor oil 60); or polyoxyethylene polyoxypropylene glycols (e.g., polyoxyethylene (42) polyoxypropylene (67) glycol, polyoxyethylene (54) polyoxypropylene (39) glycol, polyoxyethylene (105) polyoxypropylene (5) glycol, polyoxyethylene (124) polyoxypropylene (39) glycol, polyoxyethylene (160) polyoxypropylene (30) glycol, polyoxyethylene (196) polyoxypropylene (67) glycol, and polyoxyethylene (200) polyoxypropylene (70) glycol).
Item 7. The method, the combination drug, the vaccine formulation, or the kit according to any one of Items 1 to 5, wherein the hydrophilic surfactant is polysorbate 20, polysorbate 40, polysorbate 80, polyoxyethylene hydrogenated castor oil 60, or polyoxyethylene (160) polyoxypropylene (30) glycol.
Item 8. The method, the combination drug, the vaccine formulation, or the kit according to any one of Items 1 to 5, wherein the hydrophilic surfactant is polysorbate 20, polysorbate 40, or polysorbate 80.
Item 9. The method, the combination drug, the vaccine formulation, or the kit according to any one of Items 1 to 8, wherein the lipophilic surfactant is sorbitan fatty acid esters (e.g., sorbitan fatty acid ester, sorbitan monolaurate, sorbitan monopalmitate, sorbitan monostearate, sorbitan tristearate, sorbitan monooleate, sorbitan sesquioleate, sorbitan trioleate, and medium-chain triglyceride); glycerin fatty acid esters (e.g., glycerin fatty acid ester, glyceryl monostearate, glyceryl monomyristate, glyceryl monooleate, and glyceryl triisooctanoate); sucrose fatty acid esters (e.g., sucrose fatty acid ester, sucrose stearate, and sucrose palmitate); or propylene glycol fatty acid esters (e.g., propylene glycol fatty acid ester and propylene glycol monostearate).
Item 10. The method, the combination drug, the vaccine formulation, or the kit according to any one of Items 1 to 8, wherein the lipophilic surfactant is sorbitan fatty acid ester, sorbitan monooleate, sorbitan sesquioleate, or sorbitan trioleate.
Item 11. The method, the combination drug, the vaccine formulation, or the kit according to any one of Items 1 to 8, wherein the lipophilic surfactant is sorbitan trioleate.
Item 12. The method, the combination drug, the vaccine formulation, or the kit according to any one of Items 1 to 11, wherein the pharmaceutical composition further comprises an antioxidant agent B selected from the group consisting of tocopherols (e.g., α-tocopherol, β-tocopherol, γ-tocopherol, and δ-tocopherol); tocopherol acetate; and butylhydroxyanisole.
Item 13. The method, the combination drug, the vaccine formulation, or the kit according to any one of Items 1 to 11, wherein the pharmaceutical composition further comprises an antioxidant agent B selected from the group consisting of α-tocopherol, β-tocopherol, γ-tocopherol, and δ-tocopherol.
Item 14. The method, the combination drug, the vaccine formulation, or the kit according to any one of Items 1 to 11, wherein the pharmaceutical composition further comprises an antioxidant B of α-tocopherol.
Item 15. The method, the combination drug, the vaccine formulation, or the kit according to any one of Items 1 to 14, wherein the antioxidant agent A is ascorbyl palmitate, potassium ascorbate, sodium ascorbate, or ascorbic acid.
Item 16. The method, the combination drug, the vaccine formulation, or the kit according to any one of Items 1 to 14, wherein the antioxidant agent A is sodium ascorbate or potassium ascorbate.
Item 17. The method, the combination drug, the vaccine formulation, or the kit according to any one of Items 1 to 16, wherein the excipient is non-reducing sugars (e.g., sucrose and trehalose) or sugar alcohols (e.g., sorbitol, erythritol, xylitol, maltitol, and lactitol).
Item 18. The method, the combination drug, the vaccine formulation, or the kit according to any one of Items 1 to 16, wherein the excipient is sucrose, trehalose, sorbitol, or xylitol.
Item 19. The method, the combination drug, the vaccine formulation, or the kit according to any one of Items 1 to 16, wherein the excipient is sucrose or trehalose.
Item 20. The method, the combination drug, the vaccine formulation, or the kit according to any one of Items 1 to 19, wherein the content of squalane in the pharmaceutical composition ranges from 50- to 500-fold of the weight of Compound A.
Item 21. The method, the combination drug, the vaccine formulation, or the kit according to any one of Items 1 to 19, wherein the content of squalane in the pharmaceutical composition ranges from 100- to 400-fold of the weight of Compound A.
Item 22. The method, the combination drug, the vaccine formulation, or the kit according to any one of Items 1 to 19, wherein the content of squalane in the pharmaceutical composition ranges from 200- to 300-fold of the weight of Compound A.
Item 23. The method, the combination drug, the vaccine formulation, or the kit according to any one of Items 1 to 22, wherein the content of the hydrophilic surfactant in the pharmaceutical composition ranges from 0.5- to 250-fold of the weight of Compound A.
Item 24. The method, the combination drug, the vaccine formulation, or the kit according to any one of Items 1 to 22, wherein the content of the hydrophilic surfactant in the pharmaceutical composition ranges from 5- to 100-fold of the weight of Compound A.
Item 25. The method, the combination drug, the vaccine formulation, or the kit according to any one of Items 1 to 22, wherein the content of the hydrophilic surfactant in the pharmaceutical composition ranges from 10- to 50-fold of the weight of Compound A.
Item 26. The method, the combination drug, the vaccine formulation, or the kit according to any one of Items 1 to 25, wherein the content of the lipophilic surfactant in the pharmaceutical composition ranges from 0.5- to 250-fold of the weight of Compound A.
Item 27. The method, the combination drug, the vaccine formulation, or the kit according to any one of Items 1 to 25, wherein the content of the lipophilic surfactant in the pharmaceutical composition ranges from 5- to 100-fold of the weight of Compound A.
Item 28. The method, the combination drug, the vaccine formulation, or the kit according to any one of Items 1 to 25, wherein the content of the lipophilic surfactant in the pharmaceutical composition ranges from 10- to 50-fold of the weight of Compound A.
Item 29. The method, the combination drug, the vaccine formulation, or the kit according to any one of Items 1 to 28, wherein the content of the antioxidant agent A in the pharmaceutical composition ranges from 0.5- to 500-fold of the weight of Compound A when calculated in terms of the weight of sodium ascorbate.
Item 30. The method, the combination drug, the vaccine formulation, or the kit according to any one of Items 1 to 28, wherein the content of the antioxidant agent A in the pharmaceutical composition ranges from 2.5- to 250-fold of the weight of Compound A when calculated in terms of the weight of sodium ascorbate.
Item 31. The method, the combination drug, the vaccine formulation, or the kit according to any one of Items 1 to 28, wherein the content of the antioxidant agent A in the pharmaceutical composition ranges from 5- to 100-fold of the weight of Compound A when calculated in terms of the weight of sodium ascorbate.
Item 32. The method, the combination drug, the vaccine formulation, or the kit according to any one of Items 1 to 31, wherein the content of the excipient in the pharmaceutical composition ranges from 50- to 1000-fold of the weight of Compound A.
Item 33. The method, the combination drug, the vaccine formulation, or the kit according to any one of Items 1 to 31, wherein the content of the excipient in the pharmaceutical composition ranges from 100- to 750-fold of the weight of Compound A.
Item 34. The method, the combination drug, the vaccine formulation, or the kit according to any one of Items 1 to 31, wherein the content of the excipient in the pharmaceutical composition ranges from 200- to 625-fold of the weight of Compound A.
Item 35. The method, the combination drug, the vaccine formulation, or the kit according to any one of Items 12 to 34, wherein the content of the antioxidant agent B in the pharmaceutical composition ranges from 5- to 250-fold of the weight of Compound A.
Item 36. The method, the combination drug, the vaccine formulation, or the kit according to any one of Items 12 to 34, wherein the content of the antioxidant agent B in the pharmaceutical composition ranges from 12.5- to 125-fold of the weight of Compound A.
Item 37. The method, the combination drug, the vaccine formulation, or the kit according to any one of Items 12 to 34, wherein the content of the antioxidant agent B in the pharmaceutical composition ranges from 25- to 50-fold of the weight of Compound A.
Item 38. The method, the combination drug, the vaccine formulation, or the kit according to any one of Items 1 to 37, wherein the weight of Compound A ranges from 0.0001- to 0.65-fold of the weight of a lyophilized substance of the pharmaceutical composition excluding Compound A.
Item 39. The method, the combination drug, the vaccine formulation, or the kit according to any one of Items 1 to 37, wherein the weight of Compound A ranges from 0.0002- to 0.35-fold of the weight of a lyophilized substance of the pharmaceutical composition excluding Compound A.
Item 40. The method, the combination drug, the vaccine formulation, or the kit according to any one of Items 1 to 37, wherein the weight of Compound A ranges from 0.0005- to 0.065-fold of the weight of a lyophilized substance of the pharmaceutical composition excluding Compound A.
Item 41. The method, the combination drug, the vaccine formulation, or the kit according to any one of Items 5 to 40, wherein the particle size D₉₀ values of an emulsion of the pharmaceutical composition right after manufacturing and an emulsion reconstituted after storage for 6 months at 25° C. as a lyophilized formulation are 1000 nm or below.
Item 42. The method, the combination drug, the vaccine formulation, or the kit according to any one of Items 5 to 41, wherein the increased amount in the area percentage value of an impurity UK-1.02 after a lyophilized formulation of the pharmaceutical composition is stored for 6 months at 5° C. is 5.0% or below.
Item 43. The method, the combination drug, the vaccine formulation, or the kit according to any one of Items 5 to 41, wherein the increased amount in the area percentage value of an impurity UK-1.02 after a lyophilized formulation of the pharmaceutical composition is stored for 6 months at 5° C. is 1.0% or below.
Item 44. A method for blocking transmission of malaria parasites from a human to a mosquito, comprising administering a pharmaceutically effective amount of I) a pharmaceutical composition to a human residing in an area in need of blocking malaria transmission in combination with a pharmaceutically effective amount of II) a vaccine, wherein:
I) the pharmaceutical composition comprises the following ingredients i) to vi):

i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;

ii) squalane;

iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;

iv) an excipient selected from the group consisting of non-reducing sugars and sugar alcohols, except for mannitol;

v) a hydrophilic surfactant; and

vi) a lipophilic surfactant; and

II) the vaccine is a malaria vaccine comprising an antigen having the sequence represented by SEQ ID NO: 1 or SEQ ID NO: 2.
Item 45. A pharmaceutical composition for use in blocking transmission of malaria parasites in combination with a malaria vaccine, wherein:

the pharmaceutical composition comprises the following ingredients i) to vi):

i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;

ii) squalane;

iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;

iv) an excipient selected from the group consisting of non-reducing sugars and sugar alcohols, except for mannitol;

v) a hydrophilic surfactant; and

vi) a lipophilic surfactant; and

the malaria vaccine comprises an antigen having the sequence represented by SEQ ID NO: 1 or SEQ ID NO: 2.

Item 46. A malaria vaccine for use in blocking transmission of malaria parasites in combination with a pharmaceutical composition, wherein:

the malaria vaccine comprises an antigen having the sequence represented by SEQ ID NO: 1 or SEQ ID NO: 2; and

the pharmaceutical composition comprises the following ingredients i) to vi):

i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;

ii) squalane;

iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;

iv) an excipient selected from the group consisting of non-reducing sugars and sugar alcohols, except for mannitol;

v) a hydrophilic surfactant; and

vi) a lipophilic surfactant.

### Advantageous Effects of Invention

According to the present invention, combination use of a pharmaceutical composition as a vaccine adjuvant with enhanced specific immune response against antigens and good preservation stability and a malaria vaccine with non-glycosylation, homogeneity, and biological activity allows for the provision of malaria transmission-blocking vaccines with good preservation stability and immunostimulatory action.

### BRIEF DESCRIPTION OF DRAWINGS

FIG. 1 shows electrophoresis of purified Pfs230D1+. (a) SDS-PAGE and (b) anti-His Western blot analysis.

FIG. 2 shows Pfs230D1+ histidine-tagged protein induced functional antibodies in rats. (a) Antibody level in each serum was determined against corresponding immunogen by ELISA. Individual (dots) and geometric mean (bars) ELISA units are shown. (b) 250 ug/mL of total IgG from each group was tested with complement in SMFA. Transmission Reducing Activity (TRA) for each group is shown with standard error of the mean.

FIG. 3 shows electrophoresis of purified non-tagged Pfs230D1+ of SDS-PAGE analysis.

FIG. 4 shows electrophoresis of purified Pfs230D1+ and anti-Pfs230 human monoclonal antibodies Western blot analysis. (a) Pfs230D1+ containing histidine-tag reacted to two conformational dependent anti-Pfs230 monoclonal antibodies under Non-reducing conditions. (b) Non-tagged Pfs230D1+ reacted to two conformational dependent anti-Pfs230 monoclonal antibodies under Non-reducing conditions. (R) refers to reducing conditions, and (NR) refers to non-reducing conditions.

FIG. 5 shows Pfs230D1+ non-tagged protein induced functional antibodies in rats. (a) Antibody level in each serum was determined against corresponding immunogen by ELISA. Individual (dots) and geometric mean (bars) ELISA units are shown. (b) 250 ug/mL of total IgG from each group was tested with complement in SMFA. Transmission Reducing Activity (TRA) for each group is shown with standard error of the mean.

### DESCRIPTION OF EMBODIMENTS

### Pharmaceutical Composition

Pharmaceutical compositions herein include a lyophilized formulation of an emulsion comprising Compound A, squalane, an antioxidant agent A of ascorbic acids, and an excipient A. The emulsion formulation before lyophilization and a reconstituted emulsion formulation from the lyophilized formulation are also encompassed in the present invention.

In the pharmaceutical compositions, Compound A comprised in the active ingredient may be in the free form or any pharmaceutically acceptable acid-addition salts or base-addition salts thereof. Such acid-addition salts include, for example, acid-addition salts with inorganic or organic acids such as hydrochloric acid, hydrobromic acid, sulfuric acid, trifluoroacetic acid, citric acid, and maleic acid. Such base-addition salts include, for example, alkali metal salts such as sodium and potassium salts, alkaline-earth metal salts such as calcium salt, and ammonium salts. Compound A or a pharmaceutically acceptable salt thereof herein may also exist in the form of hydrates and solvates which are also included in Compound A or a pharmaceutically acceptable salt thereof herein. Details and preparations for them are described in PTL 1, and Compound A or a pharmaceutically acceptable salt thereof may be prepared according to, for example, the methods described in PTL 1.

The content of Compound A in the pharmaceutical composition is described as that of the free form of Compound A. When Compound A is used in its pharmaceutically acceptable salt, the content is calculated in terms of the weight of Compound A with addition of the weight of the salt.

Emulsions or emulsion formulations herein refer to oil-in-water type or water-in-oil type emulsions. Oil-in-water type emulsions are preferred. The ratio by weight of an oil composition to an aqueous solution ranges preferably from 1:99 to 15:85, more preferably from 2:98 to 10:90, furthermore preferably from 3:97 to 9:91, still furthermore preferably from 4:96 to 7:93. In an emulsion formulation herein, Compound A is dissolved to exist in the oil composition.

Lyophilized formulations herein refer to the formulation where water is removed from the emulsion formulation under lyophilization. The emulsion formulation may be reconstituted with two- to twenty-fold weight of water for injection to the weight of a lyophilized formulation.

The hydrophilic surfactant herein includes polyoxyethylene sorbitan fatty acid esters (e.g., polysorbate 20, polysorbate 40, polysorbate 60, polysorbate 65, and polysorbate 80); polyoxyethylene hydrogenated castor oils (e.g., polyoxyethylene hydrogenated castor oil 10, polyoxyethylene hydrogenated castor oil 20, polyoxyethylene hydrogenated castor oil 40, polyoxyethylene hydrogenated castor oil 50, and polyoxyethylene hydrogenated castor oil 60); and polyoxyethylene polyoxypropylene glycols (e.g., polyoxyethylene (42) polyoxypropylene (67) glycol, polyoxyethylene (54) polyoxypropylene (39) glycol, polyoxyethylene (105) polyoxypropylene (5) glycol, polyoxyethylene (124) polyoxypropylene (39) glycol, polyoxyethylene (160) polyoxypropylene (30) glycol, polyoxyethylene (196) polyoxypropylene (67) glycol, and polyoxyethylene (200) polyoxypropylene (70) glycol). Polysorbate 20, polysorbate 40, polysorbate 80, polyoxyethylene hydrogenated castor oil 60, and polyoxyethylene (160) polyoxypropylene (30) glycol are preferred; polysorbate 20, polysorbate 40, and polysorbate 80 are further preferred; and polysorbate 80 is particularly preferred.

The content of the hydrophilic surfactant in the pharmaceutical composition ranges from 0.5- to 250-fold of the weight of Compound A, preferably from 5- to 100-fold, more preferably from 10- to 50-fold.

The lipophilic surfactant herein includes sorbitan fatty acid esters (e.g., sorbitan fatty acid ester, sorbitan monolaurate, sorbitan monopalmitate, sorbitan monostearate, sorbitan tristearate, sorbitan monooleate, sorbitan sesquioleate, sorbitan trioleate, and medium-chain triglyceride); glycerin fatty acid esters (e.g., glycerin fatty acid ester, glyceryl monostearate, glyceryl monomyristate, glyceryl monooleate, and glyceryl triisooctanoate); sucrose fatty acid esters (e.g., sucrose fatty acid ester, sucrose stearate, and sucrose palmitate); and propylene glycol fatty acid esters (e.g., propylene glycol fatty acid ester and propylene glycol monostearate). Sorbitan fatty acid ester, sorbitan monooleate, sorbitan sesquioleate, and sorbitan trioleate are preferred; and sorbitan trioleate is further preferred.

The content of the lipophilic surfactant in the pharmaceutical composition ranges from 0.5- to 250-fold of the weight of Compound A, preferably from 5- to 100-fold, more preferably from 10- to 50-fold.

Oil compositions in the pharmaceutical composition herein include squalane. In formulation studies of the pharmaceutical compositions, squalane is preferably used for the oil composition in the pharmaceutical composition because the oxidative stability of Compound A is better in the use of squalane than that in the use of squalene commonly used as oil compositions for emulsion formulations. The content of squalane in the pharmaceutical composition ranges from 50- to 500-fold of the weight of Compound A, preferably from 100- to 400-fold, more preferably from 200- to 300-fold.

The antioxidant agent A herein includes ascorbic acid esters (e.g., L-ascorbyl stearate and ascorbyl palmitate); inorganic acid salts of ascorbic acid (e.g., potassium ascorbate, sodium ascorbate, and calcium ascorbate); and ascorbic acid. Ascorbyl palmitate, potassium ascorbate, sodium ascorbate, and ascorbic acid are preferred; and sodium ascorbate and potassium ascorbate are further preferred.

The content of the antioxidant agent A in the pharmaceutical composition ranges from 0.5- to 500-fold of the weight of Compound A, preferably from 2.5- to 250-fold, more preferably from 5- to 100-fold, wherein the content is calculated in terms of sodium ascorbate; i.e., the content is calculated by converting ascorbic acid of the antioxidant agent A, ascorbic acid derivatives, into sodium ascorbate by weight.

The excipient A herein includes non-reduced sugars and sugar alcohols (except for mannitol). Non-reduced sugars (e.g., sucrose, trehalose) and sugar alcohols (e.g., sorbitol, erythritol, xylitol, maltitol, and lactitol) are preferred; sucrose, trehalose, sorbitol, and xylitol are further preferred; sucrose and trehalose are furthermore preferred; and sucrose is particularly preferred.

The content of the excipient A in the pharmaceutical composition ranges from 50- to 1000-fold of the weight of Compound A, preferably from 100- to 750-fold, more preferably from 200- to 625-fold.

The antioxidant agent B herein includes tocopherols (e.g., α-tocopherol, β-tocopherol, γ-tocopherol, and δ-tocopherol); tocopherol acetate; and butylhydroxyanisole. α-tocopherol, β-tocopherol, γ-tocopherol, and δ-tocopherol are preferred; and α-tocopherol is further preferred.

The content of the antioxidant agent B in the pharmaceutical composition ranges from 5- to 250-fold of the weight of Compound A, preferably from 12.5- to 125-fold, more preferably from 20- to 50-fold, furthermore preferably from 25- to 50-fold.

Lyophilized formulations herein may be prepared by charging an emulsion into a vial and lyophilizing under commonly-used manufacturing conditions with a lyophilizer. Such manufacturing conditions are not limited, but specifically include, for example, the condition of freezing at around −40° C., followed by depressurizing in vacuo inside while increasing the temperature inside to −20° C. and drying for around 10 to 80 hours, then increasing the temperature inside to 25° C. and drying for around 10 to 30 hours.

One embodiment of the pharmaceutical compositions includes a lyophilized formulation of an emulsion, comprising:

i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaenamide or a pharmaceutically acceptable salt thereof;
ii) squalane;
iii) an antioxidant agent A selected from the group consisting of ascorbic acid esters (e.g., L-ascorbyl stearate and ascorbyl palmitate), inorganic salts of ascorbic acid (e.g., potassium ascorbate, sodium ascorbate, and calcium ascorbate), and ascorbic acid;
iv) an excipient A selected from the group consisting of non-reduced sugars and sugar alcohols (except for mannitol);
v) a hydrophilic surfactant; and
vi) a lipophilic surfactant.

Another embodiment of the pharmaceutical compositions includes a lyophilized formulation of an emulsion, comprising:

i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaenamide or a pharmaceutically acceptable salt thereof;
ii) squalane;
iii) an antioxidant agent A selected from the group consisting of ascorbic acid esters (e.g., L-ascorbyl stearate and ascorbyl palmitate), inorganic salts of ascorbic acid (e.g., potassium ascorbate, sodium ascorbate, and calcium ascorbate), and ascorbic acid;
iv) an excipient A selected from the group consisting of non-reduced sugars and sugar alcohols (except for mannitol);
v) a hydrophilic surfactant such as polyoxyethylene sorbitan fatty acid esters (e.g., polysorbate 20, polysorbate 40, polysorbate 60, polysorbate 65, and polysorbate 80); polyoxyethylene hydrogenated castor oils (e.g., polyoxyethylene hydrogenated castor oil 10, polyoxyethylene hydrogenated castor oil 20, polyoxyethylene hydrogenated castor oil 40, polyoxyethylene hydrogenated castor oil 50, and polyoxyethylene hydrogenated castor oil 60); and polyoxyethylene polyoxypropylene glycols (e.g., polyoxyethylene (42) polyoxypropylene (67) glycol, polyoxyethylene (54) polyoxypropylene (39) glycol, polyoxyethylene (105) polyoxypropylene (5) glycol, polyoxyethylene (124) polyoxypropylene (39) glycol, polyoxyethylene (160) polyoxypropylene (30) glycol, polyoxyethylene (196) polyoxypropylene (67) glycol, and polyoxyethylene (200) polyoxypropylene (70) glycol); vi) a lipophilic surfactant such as sorbitan fatty acid esters (e.g., sorbitan fatty acid ester, sorbitan monolaurate, sorbitan monopalmitate, sorbitan monostearate, sorbitan tristearate, sorbitan monooleate, sorbitan sesquioleate, sorbitan trioleate, and medium-chain triglyceride); glycerin fatty acid esters (e.g., glycerin fatty acid ester, glyceryl monostearate, glyceryl monomyristate, glyceryl monooleate, and glyceryl triisooctanoate); sucrose fatty acid esters (e.g., sucrose fatty acid ester, sucrose stearate, and sucrose palmitate); propylene glycol fatty acid esters (e.g., propylene glycol fatty acid ester and propylene glycol monostearate); and
iii′) an antioxidant agent B selected from the group consisting of tocopherols (e.g., α-tocopherol, β-tocopherol, γ-tocopherol, and δ-tocopherol), tocopherol acetate, and butylhydroxyanisole.

Still another embodiment of the pharmaceutical compositions includes a lyophilized formulation comprising iii′) tocopherols (e.g., α-tocopherol, β-tocopherol, γ-tocopherol, and δ-tocopherol) without comprising the antioxidant agent A of iii) for an antioxidant agent. Such tocopherols are preferably α-tocopherol.

The content of tocopherol in the pharmaceutical composition ranges from 5- to 250-fold of the weight of Compound A, preferably from 12.5- to 125-fold, more preferably from 20- to 50-fold, furthermore preferably from 25- to 50-fold.

In such a pharmaceutical composition, sodium thiosulfate or butylhydroxyanisole may also be included as an additional antioxidant agent.

The weight of Compound A in the pharmaceutical composition ranges from 0.0001- to 0.65-fold of the weight of a lyophilized substance of the pharmaceutical composition excluding Compound A, preferably from 0.0002- to 0.35-fold, more preferably from 0.0005- to 0.065-fold.

The particle size D90 value of oil droplets in the pharmaceutical composition is 1000 nm or below, preferably 300 nm or below, as the particle size D90 value of an emulsion during the manufacturing process or right after manufacturing. The emulsion right after manufacturing includes, for example, the emulsion within 30 seconds after manufactured. The particle size D₉₀ value of oil droplets of an emulsion reconstituted after storage as a lyophilized formulation is preferably 1000 nm or below as the particle size D₉₀ value of oil droplets of an emulsion reconstituted after storage for 6 months at 5° C. or 25° C.

In the pharmaceutical compositions, the particle size D₉₀ value of oil droplets is a typical value that shows the particle-size distribution of oil droplet particles comprised in an emulsion and refers to a 90% particle size based on the scattering intensity. In general, particle size D₉₀ values are measured and calculated with a dynamic-light-scattering particle-size distribution analyzer, laser-diffraction particle-size analyzer, or image-processing particle-size distribution analyzer. The particle size D₉₀ values herein refer to those measured with a dynamic-light-scattering particle-size distribution analyzer: Zetasizer Nano ZS (Malvern Instruments).

In the pharmaceutical compositions, an impurity UK-1.02 is one of typical impurities detected in the assessment of related substances with a high-performance liquid chromatograph. In particular, it refers to the impurity detected at the 1.02-fold elution time of Compound A in the spectrographic measurement with a 220-nm wavelength by reverse-phase high-performance liquid chromatography using pure water, acetonitrile, methanol, and trifluoroacetic acid with a Phenyl-Hexyl column (Waters Xselect CSH Phenyl-Hexyl XP Column, 4.6 mm×75 mm, 2.5 μm, model number: 186006134) injecting 0.4 to 2 μg calculated as the content of Compound A. Details of the measurement conditions are as follows. Mobile phase A: 0.1% aqueous trifluoroacetic acid solution Mobile phase B: acetonitrile/methanol mixed solution (8:2) containing 0.06% trifluoroacetic acid

### Gradient Conditions:

TABLE 1

| Time (min.) | Mobile phase A:Mobile phase B |
|---|---|
| 0.0 to 0.5 | 6:4 |
| 0.5 to 50.5 | 6:4 -> 1:9 |
| 50.5 to 65.0 | 1:9 |
| 65.0 to 65.1 | 1:9 -> 6:4 |
| 75.0 | 6:4 |

Flow rate: 0.5 mL/min.
Column temperature: Constant temperature at around 40° C.

The preservation stability of the pharmaceutical composition means that the increased amount in the area percentage value of an impurity UK-1.02 after a lyophilized formulation of the pharmaceutical composition is stored for 6 months at 5° C. is 5.0% or below, preferably 1.0% or below, of the value at the start of storage. The area percentage values are compared in the actual measured values.

The pharmaceutical composition is stored in the lyophilized condition where the oil-in-water emulsion prepared is emulsified, followed by aseptic filtration with an aseptic filtration filter. In the aseptic filtration, the particle size D₉₀ value is preferably 1000 nm or below so as to avoid clogging and allow for efficient filtration.

The pharmaceutical composition may further comprise additional additives as long as the particle size of the emulsion after reconstitution is unchanged. When administered, the pharmaceutical composition may be administered in combination with a formulation comprising a vaccine antigen, also referred to as a “vaccine” herein, as long as the particle size of the emulsion after reconstitution is unchanged. Mixing methods and ratios of the pharmaceutical composition and such a vaccine antigen are not limited, but for example, a formulation comprising a vaccine antigen may be combined by inversion mixing in a vial in the same volume as that of the reconstituted emulsion formulation.

The pharmaceutical composition may be provided as a kit comprising a lyophilized formulation comprising Compound A and a vaccine antigen.

The pharmaceutical composition may be administered by reconstitution with 2- to 20-fold of water for injection by weight of a lyophilized formulation when administered, followed by mixing with a formulation comprising a vaccine antigen. A dosage amount of the pharmaceutical composition is 1 ng to 250 mg, preferably 1 ng to 50 mg, of the weight of Compound A per dose. The administration may be in a single dose or with one or more additional doses depending on the kind of the vaccine antigen simultaneously administered or the age of the subject to be administered.

The preservation stability of pharmaceutical compositions was assessed according to the test examples below.

### Vaccine Antigen

One embodiment of the vaccine antigen in the present invention includes a malaria vaccine comprising an N-terminal Pfs230 antigen. In another embodiment, the vaccine antigen is a vaccine comprising an antigen of Pfs230D1+ having the sequence represented by SEQ ID NO: 1 (also referred to as “non-tagged Pfs230D1+” herein). In still another embodiment, the vaccine antigen is a vaccine comprising an antigen of Pfs230D1+ having the sequence represented by SEQ ID NO: 2 (also referred to as “tagged Pfs230D1+” herein).

A scalable baculovirus expression system may be used to express the Pfs230D1+ construct, which is subsequently purified and analysed. Pfs230D1+ may be designed to avoid glycosylation and protease digestion, thereby potentially increasing homogeneity and stability. In particular, Pfs230D1+ may eliminate the undesirable glycosylation as well as resulting in a two-fold increase in yield and increased stability.

### Combination Drug/Combination Therapy

In malaria transmission-blocking vaccines in the present invention, combination use of a vaccine antigen and a pharmaceutical composition comprising an adjuvant, Compound A, having a TLR7 agonist activity may enhance the inducing property of IgG2 antibody to show an improved vaccine activity. Such combination use may include administering the vaccine antigen and the pharmaceutical composition simultaneously or separately with a prescribed time interval. In one embodiment, such simultaneous combination use includes a combination drug comprising the vaccine antigen and the pharmaceutical composition. Such a combination drug may also be referred to as an “adjuvant formulation” or a “vaccine formulation” herein. In another embodiment, the combination use includes a kit comprising the vaccine antigen and the pharmaceutical composition.

Administration routes of a pharmaceutical composition, a vaccine antigen, a combination drug, and a kit herein may be selected depending on conditions such as diseases, conditions of subjects, and target sites. Such administration routes include, for example, parenteral administration, specifically, intravascular such as intravenous, subcutaneous, intracutaneous, intramuscular, transnasal, and transdermal administration.

Dosage forms of a pharmaceutical composition, a vaccine antigen, and a combination drug herein include, for example, injections such as prefilled syringes.

Doses, dosage regimens, and time required for each administration of adjuvant formulations herein may be selected depending on conditions such as ages of subjects and target sites. Such adjuvant formulations may be administered or innoculated once, or may be further administered in a prescribed time period after first administration. The time period from the first administration to additional administration may, for example, be any period from 20 days to 3 years, preferably from 3 months to 2 years, more preferably from 6 months to 1 year, but is not limited thereto.

A dosage amount of a malaria transmission-blocking vaccine antigen per each dose in combination use herein may range from 1 μg to 200 μg, preferably from 10 μg to 30 μg, more preferably 15 μg, but is not limited thereto. One dose of an adjuvant formulation includes, for example, 0.5 mL.

A dosage amount of a pharmaceutical composition comprising Compound A per each dose in combination use herein may range from 1 ng to 250 mg, preferably from 1 ng to 50 mg, of the weight of Compound A, but is not limited thereto.

### EXAMPLES

Hereinafter, the present invention is illustrated with Examples, Reference examples, Comparative examples, and Test examples, but is not intended to be limited thereto.

Herein, “squalane (Wako pure chemical)”, “squalane (Kishimoto Special Liver Oil Co., Ltd.)”, or “squalane (Maruha Nichiro)” was used for squalane; “squalene (Wako pure chemical)”, “squalene (Kishimoto Special Liver Oil Co., Ltd.)”, or “squalene (Maruha Nichiro)” was used for squalene; “sodium ascorbate (Wako pure chemical)” or “sodium L-ascorbate (Kyowa Pharma Chemical Co., Ltd.)” was used for sodium ascorbate; “α-tocopherol (Mitsubiti-Chemical Foods Corporation)” or “all-rac-α-Tocopherol EMPROVE (registered trade mark) ESSENTIAL Ph Eur,BP,USP,E 307 (Merck)” was used for α-tocopherol; “Span85 (Sigma-Aldrich)”, “Rheodol SP-030V (Kao Chemicals)”, or “Span85 (CRODA)” was used for sorbitan trioleate; “PS80 (GS) (NOF Corporation)”, “Polysorbate 80 (HX2) (NOF Corporation)”, “Tween 80 (Merck)”, or “Tween 80 HP-LQ-(HM) (CRODA)” was used for polysorbate 80; “sucrose (Nacalai Tesque)” or “sucrose low in endotoxins suitable for use as excipient EMPROVE (registered trade mark) exp Ph Eur,BP,JP,NF (Merck)” was used for sucrose; “OTSUKA distilled water for injection (Otsuka Pharmaceutical Factory)” was used for water for injection; ascorbyl palmitate, butylhydroxyanisole, and sodium thiosulfate were prepared from Wako pure chemical for use.

### Abbreviations

ACN: acetonitrile

### AAALAC: Association for Assessment and Accreditation of

### Laboratory Animal Care

BCA: bicinchoninic acid
cIEF: capillary isoelectric focusing
CM 1: cysteine motif 1
CV: column volume

### Da: Dalton

DNA: deoxyribonucleic acid
DTT: dithiothreitol
ELISA: enzyme-linked immunosorbent assay
EPA: Pseudomonas aeruginosa exoprotein A
HPLC: high performance liquid chromatography

### IACUC: Institutional Animal Care and use Committee

kDa: kilodalton
LDS: Lithium dodecyl sulfate
MES: 2-(N-morpholino) ethanesulfonic acid
MOI: multiplicity of infection
MS: mass spectrometry
MS1: Mass spectral
Ni-NTA: Nickel-nitrilotriacetic acid

### NIAID: National Institute of Allergy and Infectious Diseases

### NIH: National Institutes of Health

### NLS: Noble Life Sciences

### OLAW: Office of Laboratory Animal Welfare

RP: reversed-phase
SDS: sodium dodecyl sulfate
SDS-PAGE: sodium dodecyl sulfate polyacrylamide gel
electrophoresis
SE: size exclusion
SEC: size exclusion chromatography
SMFA: standard membrane feeding assay
TBV: transmission-blocking vaccine
TFA: trifluoroacetic acid
TRA: transmission reducing activity
UHPLC: ultrahigh performance liquid chromatography

### USDA: United States Department of Agriculture

ZINB: zero-inflated negative bionomial

### Preparation of Lyophilized Compositions

### Examples 1 to 16, Comparative Examples 1 to 3

Compound A was dissolved in oil-based components so as to be prepared in the compositions of Tables 1 to 4. The oil-based components are as follows: squalane, sorbitan trioleate, and α-tocopherol (Examples 1 to 9 and 15); squalane and sorbitan trioleate (Examples 10 to 13); squalane, sorbitan trioleate, α-tocopherol, and ascorbyl palmitate (Example 14); squalane, sorbitan trioleate, α-tocopherol, and butylhydroxyanisole (Example 16); squalene, sorbitan trioleate, and α-tocopherol (Comparative examples 1 to 3). Aqueous components (sucrose and polysorbate 80 in Examples 1 to 3, 14, and 16; sucrose, polysorbate 80, and sodium ascorbate in Examples 4 to 13; sucrose, polysorbate 80, and sodium thiosulfate in Example 15) were dissolved in water for injection so as to be prepared in the compositions of Tables 1 to 4, and then thereto added the above-mentioned oil-based composition. The mixture was mixed preliminarily, and emulsified to be dispersed with a ultrahigh-pressure homogenizer. The resultant was filtered through a 0.2-μm sterilizing filter, and then charged into a glass vial per 1 mL for lyophilization. Each vial was purged with nitrogen gas at ordinary pressure, and then sealed with a rubber plug to give each lyophilized composition, Examples 1 to 16 and Comparative examples 1 to 3.

TABLE 2
Table 1

| Component | Ex. 1 | Ex. 2 | Ex. 3 | Ex. 4 | Ex. 5 | Ex. 6 | Ex. 7 |
|---|---|---|---|---|---|---|---|
| Compound A | 1 | 1 | 1 | 1 | 1 | 1 | 1 |
| squalane | 225 | 200 | 237.5 | 225 | 225 | 225 | 225 |
| α-tocopherol | 25 | 50 | 12.5 | 25 | 25 | 25 | 25 |
| sodium ascorbate | 0 | 0 | 0 | 2.5 | 5 | 10 | 20 |
| ascorbyl palmitate | 0 | 0 | 0 | 0 | 0 | 0 | 0 |
| sorbitan trioleate | 25 | 25 | 25 | 25 | 25 | 25 | 25 |
| polysorbate 80 | 25 | 25 | 25 | 25 | 25 | 25 | 25 |
| sucrose | 500 | 500 | 500 | 500 | 500 | 500 | 500 |

Note:
Each value means the weight ratio to 1 part by weight of Compound A.

TABLE 3
Table 2

| Component | Ex. 8 | Ex. 9 | Ex. 10 | Ex. 11 | Ex. 12 | Ex. 13 | Ex. 14 |
|---|---|---|---|---|---|---|---|
| Compound A | 1 | 1 | 1 | 1 | 1 | 1 | 1 |
| squalane | 225 | 225 | 250 | 250 | 250 | 250 | 225 |
| α-tocopherol | 25 | 25 | 0 | 0 | 0 | 0 | 25 |
| sodium ascorbate | 40 | 60 | 10 | 20 | 40 | 60 | 0 |
| ascorbyl palmitate | 0 | 0 | 0 | 0 | 0 | 0 | 0.5 |
| sorbitan | 25 | 25 | 25 | 25 | 25 | 25 | 25 |
| trioleate | | | | | | | |
| polysorbate 80 | 25 | 25 | 25 | 25 | 25 | 25 | 25 |
| sucrose | 500 | 500 | 500 | 500 | 500 | 500 | 500 |

Note:
Each value means the weight ratio to 1 part by weight of Compound A.

TABLE 4
Table 3

| Component | Ex. 15 | Ex. 16 |
|---|---|---|
| Compound A | 1 | 1 |
| squalane | 225 | 225 |
| α-tocopherol | 25 | 25 |
| sodium ascorbate | 0 | 0 |
| sodium thiosulfate | 5 | 0 |
| butylhydroxyanisole | 0 | 0.25 |
| sorbitan trioleate | 25 | 25 |
| polysorbate 80 | 25 | 25 |
| sucrose | 500 | 500 |

Note:
Each value means the weight ratio to 1 part by weight of Compound A.

TABLE 5
Table 4

| | Comparative | Comparative | Comparative |
|---|---|---|---|
| Component | example 1 | example 2 | example 3 |
| Compound A | 1 | 1 | 1 |
| squalene | 237.5 | 225 | 200 |
| squalane | 0 | 0 | 0 |
| α-tocopherol | 12.5 | 25 | 50 |
| sodium ascorbate | 0 | 0 | 0 |
| sorbitan trioleate | 25 | 25 | 25 |
| polysorbate 80 | 25 | 25 | 25 |
| sucrose | 500 | 500 | 500 |

Note:
Each value means the weight ratio to 1 part by weight of Compound A.

### [Test Example 1] Assessment for Particle Sizes in the Manufacturing Process

In the manufacturing process of a lyophilized composition, the particle-size distribution of the oil droplet particles comprised in the emulsion after emulsification and before lyophilization was measured according to the following method.

Emulsions were diluted with water for injection to 10-fold, and the 90% particle size (D₉₀) on the basis of the scattering intensity was measured with a dynamic-light-scattering particle-size distribution analyzer (Zetasizer Nano ZS). D₉₀ (nm) for Examples 1 to 16 and Comparative examples 1 to 3 are shown in Table 5.

TABLE 6
Table 5

| | Ex. 1 | Ex. 2 | Ex. 3 | Ex. 4 | Ex. 5 | Ex. 6 | Ex. 7 | Ex. 8 |
|---|---|---|---|---|---|---|---|---|
| D₉₀ | 294 | 276 | 274 | 273 | 287 | 293 | 270 | 250 |
| (nm) | | | | | | | | |
| | Ex. 9 | Ex. 10 | Ex. 11 | Ex. 12 | Ex. 13 | Ex. 14 | Ex. 15 | Ex. 16 |
| D₉₀ | 259 | 280 | 288 | 279 | 266 | 289 | 290 | 295 |
| (nm) | | | | | | | | |

| | Comparative | | |
|---|---|---|---|
| | example 1 | Comparative example 2 | Comparative example 3 |
| D₉₀ | 280 | 309 | 273 |
| (nm) | | | |

### [Test Example 2] Assessment for Stability (1)

The particle-size distributions for the lyophilized compositions prepared were measured at the start of storage and after 6-month storage in a constant-temperature room at 5° C. and 25° C. according to the following method.

1 mL of water for injection was added to each vial of the lyophilized compositions prepared in Examples 1 to 16 and Comparative examples 1 to 3 for reconstitution. Then, 100 μL of the reconstituted solution was taken by micropipette and mixed with 900 μL of water for injection. Then, the 90% particle size (D₉₀) on the basis of the scattering intensity was measured with a dynamic-light-scattering particle-size distribution analyzer (Zetasizer Nano ZS). D₉₀ (nm) for Examples 1 to 16 and Comparative examples 1 to 3 before and after storage are shown in Table 6.

TABLE 7
Table 6

| | Ex. 1 | Ex. 2 | Ex. 3 | Ex. 4 | Ex. 5 | Ex. 6 | Ex. 7 | Ex. 8 |
|---|---|---|---|---|---|---|---|---|
| At the | 655 | 915 | 738 | 594 | 586 | 624 | 594 | 565 |
| start | | | | | | | | |
| 5° C. 6 M | 598 | 618 | 553 | 575 | 590 | 792 | 558 | 529 |
| 25° C. 6 M | 3300 | 1810 | 4650 | 772 | 599 | 946 | 606 | 502 |
| | Ex. 9 | Ex. 10 | Ex. 11 | Ex. 12 | Ex. 13 | Ex. 14 | Ex. 15 | Ex. 16 |
| At the | 564 | 662 | 621 | 562 | 554 | 665 | 698 | 694 |
| start | | | | | | | | |
| 5° C. 6 M | 600 | 610 | 601 | 508 | 544 | 508 | 585 | 576 |
| 25° C. 6 M | 937 | 594 | 551 | 488 | 477 | 505 | 837 | 1150 |

| | | | Comparative |
|---|---|---|---|
| | Comparative example 1 | Comparative example 2 | example 3 |
| At the | 675 | 552 | 1020 |
| start | | | |
| 5° C. 6 M | 579 | 631 | 625 |
| 25° C. 6 M | 5880 | 969 | 1040 |

In the test results, Examples 4 to 14 formulations containing ascorbic acids as an antioxidant agent showed higher stability of the particle-size distribution after the 6-month storage in a constant temperature room at 5° C. and 25° C. with few changes from the values at the start of storage.

### [Test Example 3] Assessment for Stability (2)

The impurities amounts (the area percentage value of Uk-1.02) were measured at the start of storage and after 6-month storage in a constant temperature room at 5° C. according to the following method. The amounts were detected by spectrographic measurement with a 220-nm wavelength by reverse-phase high-performance liquid chromatography using pure water, acetonitrile, methanol, and trifluoroacetic acid with a Phenyl-Hexyl column (Waters Xselect CSH Phenyl-Hexyl XP Column, 4.6 mm×75 mm, 2.5 μm, model number: 186006134) injecting 0.4 to 2 μg as the content of Compound A. The details of the measurement conditions are as follows. Mobile phase A: 0.1% aqueous trifluoroacetic acid solution Mobile phase B: acetonitrile/methanol mixed solution (8:2) containing 0.06% trifluoroacetic acid

### Gradient Conditions:

TABLE 8

| Time (min.) | Mobile phase A:Mobile phase B |
|---|---|
| 0.0 to 0.5 | 6:4 |
| 0.5 to 50.5 | 6:4 -> 1:9 |
| 50.5 to 65.0 | 1:9 |
| 65.0 to 65.1 | 1:9 -> 6:4 |
| 75.0 | 6:4 |

Flow rate: 0.5 mL/min.
Column temperature: Constant temperature at around 40° C.

The area percentage values of the impurities peak (Uk-1.02) detected at the 1.02-fold elution time of Compound A were calculated by the following equation with the peak areas and elution times measured in this method.

Area percentage value of Uk-1.02(%)=Peak area of Uk-1.02/Total peak area of related substances and Compound A×100

The area percentage values (%) of the impurities peak (Uk-1.02) in Examples 1 to 16 and Comparative examples 1 to 3 before and after storage are shown in Table 7.

TABLE 9
Table 7

| | Ex. 1 | Ex. 2 | Ex. 3 | Ex. 4 | Ex. 5 | Ex. 6 | Ex. 7 | Ex. 8 |
|---|---|---|---|---|---|---|---|---|
| At the start | 1.3 | 0.9 | <0.1 | 0.9 | 0.8 | 0.9 | 0.8 | <0.1 |
| 5° C. 6 M | 2.4 | 2.4 | 4.6 | 1.0 | 0.9 | 0.9 | 0.8 | 0.6 |
| | Ex. 9 | Ex. 10 | Ex. 11 | Ex. 12 | Ex. 13 | Ex. 14 | Ex. 15 | Ex. 16 |
| At the start | 0.6 | <0.1 | <0.1 | <0.1 | <0.1 | 0.7 | 1.7 | 1.4 |
| 5° C. 6 M | 0.5 | 0.3 | 0.3 | 0.4 | 0.4 | 0.8 | 3.7 | 2.8 |

| | Comparative example 1 | Comparative example 2 | Comparative example 3 |
|---|---|---|---|
| At the start | 1.0 | 0.8 | 1.1 |
| 5° C. 6 M | 27.1 | 9.4 | 7.7 |

In the test results, Examples 4 to 14 formulations containing ascorbic acids as the antioxidant agent showed higher preservation stability with lower values of impurities (the area percentage value of Uk-1.02) after the 6-month storage in a constant-temperature room at 5° C. In comparison between Examples 1 to 3 formulations containing squalane as the oil-based component and Comparative examples 1 to 3 formulations containing squalene as the oil-based component, the formulations comprising squalane as the oil-based component had lower impurities values (the area percentage value of Uk-1.02), which shows that squalane contributes to the antioxidant stability of Compound A and the formulation comprising squalane may show high reservation stability.

### Preparation of Vaccine Antigens

### [Example 17] Preparation and Purification of Pfs230D1+

### Baculovirus Expression Construct (Pfs230D1+)

The N-terminal sequence (aa 552-731) of the gametocyte surface protein Pfs230 of 3D7 strain (ACCESSION P68874), containing four cysteines as part of a predicted cysteine-rich domain, was cloned and denoted as Pfs230D1+. Codon optimization for baculovirus expression was performed by DNA2.0 (now ATUM). Synthetic deoxynucleic acid (DNA) of Pfs230D1+(552-731) contained a N585Q mutation to remove a potential N-glycosylation site, an N-terminal secretion signal (MKFLVNVALVFMVVYISYIYAD from Honeybee Melittin) and a C-terminal six histidine tag. Resulting plasmid was cloned, sequence verified, and recombinant bacmids generated as described in NPL 1. This bacmid was sequence verified again and used to transfect super Sf9 cells (Oxford Expression Technologies) for the generation of recombinant baculovirus stock using Cellfectin® II reagent (Invitrogen) following Bac-to-Bac manual.

### Expression and Purification of Pfs230D1+

Super Sf9 cells were seeded at 1×10⁶ cells/mL in SFM4 medium (Hyclone). MOI (multiplicity of infection) of one was used to infect a 10 L super Sf9 wave culture. At 96 h post infection, culture was harvested, concentrated five-fold and diafiltered with 20 mM sodium phosphate, 150 mM NaCl, pH 7.4 (Buffer A) using a tangential flow filtration device (Centrasette LV, Pall) with 0.5 m² Omega polyethersulfone membrane (Pall). Clarification was carried out with 0.22 μm filtration (Stericup-GP vacuum filter, Merck Millipore).

Two litres of clarified, concentrated supernatant was loaded onto a 61 mL (2.6×11.5 cm) Nickel-Nitrilotriacetic acid (Ni-NTA) (His60 Ni Superflow, Clontech) column at 100 cm/h. The wash steps were performed with five column volume (CV) of Buffer A, five CV of buffer A with 10 mM imidazole, then five CV of Buffer A with 20 mM imidazole. The protein was eluted with Buffer A containing 50-100 mM imidazole. Pooled eluents from Ni-NTA column were concentrated five-fold with Amicon Ultra-15 centrifugal filters, using 3K regenerated cellulose membrane (Merck Millipore). The protein was further purified, and buffer exchanged with size exclusion chromatography (SEC) on a Superdex 75 (2.6×60 cm, 320 mL), equilibrated with 20 mM HEPES, 150 mM NaCl, 5% glycerol, pH 7.2. To maintain a 5% CV injection load limit, multiple cycles were performed. The Superdex 75 elution fractions were evaluated by sodium dodecyl sulfate polyacrylamide gel electrophoresis (SDS-PAGE), pooled, and further concentrated to a target of 1 mg/mL using the same Amicon 3K concentrator (Merck Millipore) and stored at −80° C.

### Protein Concentration Determination

The bicinchoninic acid (BCA) assay was performed according to manufacturer's instruction (Thermo) to determine protein concentration of purified Pfs230D1+, i.e., tagged Pfs230D1+, due to low tryptophan content. Bovine serum albumin was used to generate the protein concentration standard curve in the assay.

### SDS-PAGE

Samples were diluted with 4×LDS (Lithium dodecyl sulfate, Invitrogen) sample buffer, heated for 5 min at 90° C. and loaded in a final volume of 20 μL/well on SDS-PAGE gels (4-12% NuPAGE Bis-Tris, Invitrogen). Gels were run at 150-200 V for 35-50 min in 1×2-(N-morpholino) ethanesulfonic acid (MES) sodium dodecyl sulfate (SDS) running buffer and stained with SimplyBlue™ SafeStain (Invitrogen).

Western Blotting (Anti-his) with Purified Pfs230D1+

Following SDS-PAGE, proteins were transferred onto nitrocellulose membrane and Western blot procedure using anti-penta His antibody (Qiagen) according to the method in NPL 1. The membrane was developed using ECL Prime (GE Healthcare).

Western Blotting (Anti-Pfs230D1+) with Purified Pfs230D1+

Following SDS-PAGE, proteins were transferred onto nitrocellulose membrane and Western blot procedure using anti-Pfs230 human monoclonal antibodies (non patent related). The human monoclonal antibodies were tested for reactivity at 10 μg/mL to 20 ng of Pfs230D1+(non-tagged or tagged) loaded per lane. A secondary goat anti-human antibody was used at 1/2000 dilution followed by development with BCIP/NBT substrate.

### N-Terminal Domain of Pfs230 (Pfs230D1+)

The new design of a Pfs230 N-terminal construct containing aa 552-731 was based on the Pfs230C1 TBV antigen (aa 443-731), denoted as Pfs230D1+, and evaluated in the super Sf9/baculovirus system. This design was selected to reduce the potential for proteolytic cleavage and glycosylation while preserving the complete disulfide-linked folding predicted to be required for induction of transmission-blocking antibodies following immunization. Pfs230D1+ also contains an additional N585Q mutation to eliminate potential N-glycosylation and a six histidine C-terminal tag to facilitate purification in the initial evaluation here. Non-tagged Pfs230D1+ contains an additional N585Q mutation to eliminate potential N-glycosylation and is absent any extraneous C-terminal amino acids. Moreover, the removal of N-terminal amino acids from the original Pfs230C1 protein did not appear to alter the predicted secondary structure of Pfs230D1+ as suggested by POLYVIEW-2D.

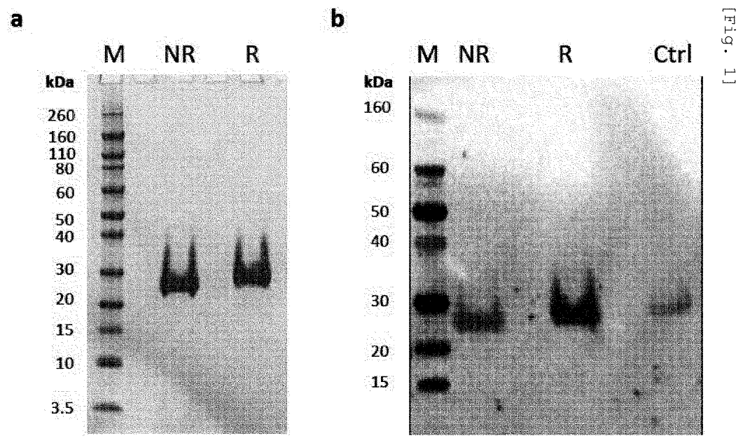

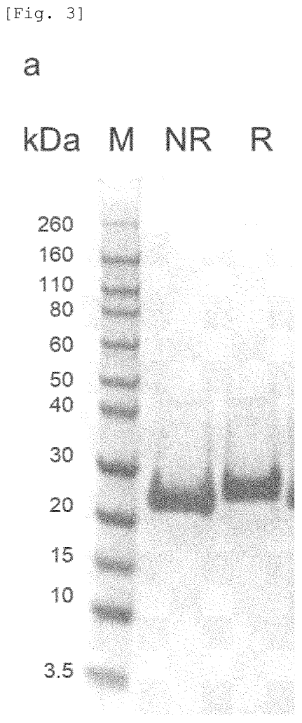

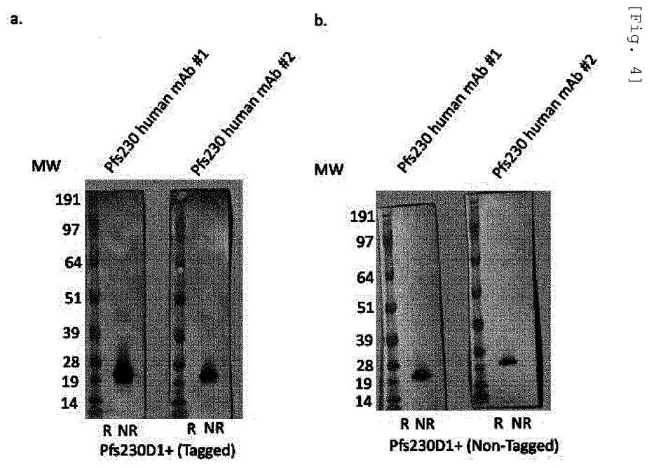

A two-step purification approach was used to capture and polish Pfs230D1+ including IMAC (immobilized metal affinity column) and SEC and resulted in a yield of 23 mg/L. Further, the resulting purified protein was present at the expected molecular weight of −21 kDa (kilodalton) and with greater than 90% purity by SDS-PAGE and densitometry (FIG. 1a). Reactivity to anti-His antibody confirmed the presence of the histidine tag on the C-terminus (FIG. 1b). A purification approach was used to capture and polish non-tagged Pfs230D1+ including IEX (ion exchange chromatography) and MMC (Mixed mode chromatography). Purified protein was desalted and buffer exchanged resulting in a final purified yield of 5-10 mg/L and with greater than 90% purity by SDS-PAGE (FIG. 3), with most impurities product related. Reactivity to anti-Pfs230 monoclonal antibodies confirmed the presence of conformational epitopes for both tagged Pfs230D1+(FIG. 4a) and non-tagged Pfs230D1+(FIG. 4b). Here (FIG. 4), both tagged Pfs230D1+ and non-tagged Pfs230D1+ reacted under non-reducing conditions (disulfide dependency) to two human Pfs230 monoclonal antibodies demonstrating the protein contained proper disulfide folding.

### Generation of Rat Anti-Pfs230 Antiserum and Enzyme-Linked Immunosorbent Assay (ELISA)

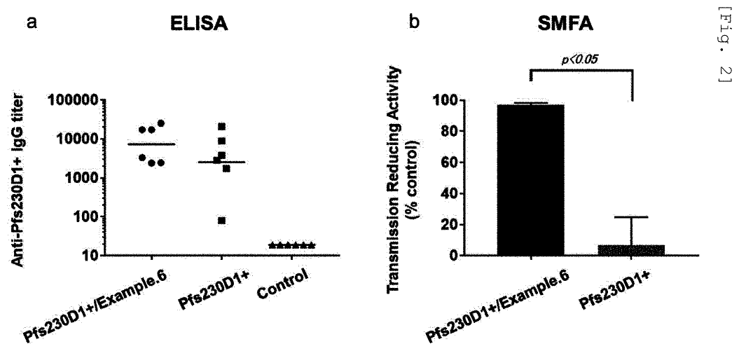

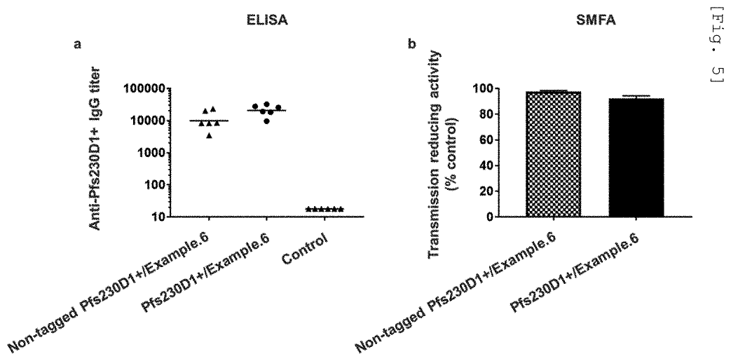

SD rats (Charles River Laboratories Japan) were immunized intramuscularly (i.m.) in the thigh muscles of the hind limb with 50 μL of a vaccine formulation consisting of 20 μg of an antigen, tagged Pfs230D1+ or non-tagged Pfs230D1+, and 8110 μg of the composition of Example 6 as an adjuvant (which corresponded to 10 μg of Compound A); 20 μg of tagged Pfs230D1+ alone for FIG. 2 or 20 μg of tagged Pfs230D1+ and 8110 μg of the composition of Example 6 for FIG. 5; or 8110 μg of the composition of Example 6 alone as a control. Rats received two or three injections on day 0 and 21 or day 0, 21, and 42. On day 42 or 56, rats were sacrificed, and blood was collected. After collection of the whole blood, the blood allowed to clot by leaving it undisturbed at room temperature for 4 hours. The clot was removed by centrifuging at 2,000×g for 10 minutes in a refrigerated centrifuge.

For the sera on day 42 or 56, the antibody levels against corresponding proteins were determined individually by ELISA (FIG. 2a for tagged Pfs230D1+, FIG. 5a for non-tagged Pfs230D1+).

### IgG Purification and Standard Membrane Feeding Assay (SMFA)

For each group, an equal amount of serum from each rat was pooled regardless of ELISA units. Total IgGs from the pooled serum sample was purified using Protein G columns (GE Healthcare) according to the manufacturer's instructions and adjusted to a final concentration of 4 mg/mL in phosphate buffered saline.

The standardized methodology for performing the SMFA is known in the art. Briefly, P. falciparum NF54 line was cultured for 16-18 days to induce mature stage V gametocytes. The stage V gametocytes (˜1% stage V gametocytemia) were mixed with test IgGs at 250 μg/mL, and the final mixture was immediately fed to ˜50 female Anopheles stephensi through a membrane-feeding apparatus. All feeding experiments were performed with human complement. Mosquitoes were kept for eight days after feeding and dissected (n=20 per group) to enumerate the oocysts in the midgut. Only midguts from mosquitoes with any eggs in their ovaries at the time of dissection were analysed (FIG. 2b for tagged Pfs230D1+, FIG. 5b for non-tagged Pfs230D1+). The human serum and red blood cells used for the gametocyte cultures and feeding experiments were purchased from Interstate Blood Bank (Memphis, Tenn.).

### INDUSTRIAL APPLICABILITY

The pharmaceutical composition comprising Compound A, or a pharmaceutically acceptable salt thereof, can show high preservation stability and immunostimulatory action as a vaccine adjuvant, and combination use of the composition with a malaria vaccine may be useful for blocking transmission of malaria parasites.

[Sequence Listing Free Text]
SEQ ID NO: 1
(Pfs230D1+; non-tagged)
DVGVDELDKI DLSYETTESG DTAVSEDSYD KYASQNTNKE
YVCDFTDQLK PTESGPKVKK CEVKVNEPLI KVKIICPLKG
SVEKLYDNIE YVPKKSPYVV LTKEETKLKE KLLSKLIYGL
LISPTVNEKE NNFKEGVIEF TLPPVVHKAT VFYFICDNSK
TEDDNKKGNR GIVEVYVEPY G
SEQ ID NO: 2
(Pfs230D1+, intermediate; tagged with histidine)
DVGVDELDKI DLSYETTESG DTAVSEDSYD KYASQNTNKE
YVCDFTDQLK PTESGPKVKK CEVKVNEPLI KVKIICPLKG
SVEKLYDNIE YVPKKSPYVV LTKEETKLKE KLLSKLIYGL
LISPTVNEKE NNFKEGVIEF TLPPVVHKAT VFYFICDNSK
TEDDNKKGNR GIVEVYVEPY G--HHHHHH
[Sequence Listing]
675859SEQ.txt

1. A method for blocking transmission of malaria parasites from a human to a mosquito, comprising administering a pharmaceutically effective amount of a combination of I) a pharmaceutical composition and II) a vaccine to a human residing in an area in need of blocking malaria transmission, wherein:
I) the pharmaceutical composition comprises the following ingredients i) to vi):
i) (4E,8E,12E,16E,20E)-N-{2-({4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide, hereinafter referred to as “Compound A”, or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and
II) the vaccine is a malaria vaccine comprising an antigen having the sequence represented by SEQ ID NO: 1 or SEQ ID NO: 2.

2. A combination drug comprising:
I) a pharmaceutical composition comprising:
i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and
II) a malaria vaccine comprising an antigen having the sequence represented by SEQ ID NO: 1 or SEQ ID NO: 2.

3. A vaccine formulation for malaria comprising:
I) a pharmaceutical composition comprising:
i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and
II) a malaria vaccine comprising an antigen having the sequence represented by SEQ ID NO: 1 or SEQ ID NO: 2.

4. A kit comprising:
I) a pharmaceutical composition comprising:
i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and
II) a malaria vaccine comprising an antigen having the sequence represented by SEQ ID NO: 1 or SEQ ID NO: 2.

5. The method according to claim 1 or the combination drug according to claim 2, wherein the pharmaceutical composition is an oil-in-water type emulsion formulation or a lyophilized formulation thereof.

6. The method according to claim 1 or the combination drug according to claim 2, wherein the hydrophilic surfactant is polyoxyethylene sorbitan fatty acid esters (e.g., polysorbate 20, polysorbate 40, polysorbate 60, polysorbate 65, and polysorbate 80); polyoxyethylene hydrogenated castor oils (e.g., polyoxyethylene hydrogenated castor oil 10, polyoxyethylene hydrogenated castor oil 20, polyoxyethylene hydrogenated castor oil 40, polyoxyethylene hydrogenated castor oil 50, and polyoxyethylene hydrogenated castor oil 60); or polyoxyethylene polyoxypropylene glycols (e.g., polyoxyethylene (42) polyoxypropylene (67) glycol, polyoxyethylene (54) polyoxypropylene (39) glycol, polyoxyethylene (105) polyoxypropylene (5) glycol, polyoxyethylene (124) polyoxypropylene (39) glycol, polyoxyethylene (160) polyoxypropylene (30) glycol, polyoxyethylene (196) polyoxypropylene (67) glycol, and polyoxyethylene (200) polyoxypropylene (70) glycol).

7. The method according to claim 1 or the combination drug according to claim 2, wherein the hydrophilic surfactant is polysorbate 20, polysorbate 40, or polysorbate 80.

8. The method according to claim 1 or the combination drug according to claim 2, wherein the lipophilic surfactant is sorbitan fatty acid esters (e.g., sorbitan fatty acid ester, sorbitan monolaurate, sorbitan monopalmitate, sorbitan monostearate, sorbitan tristearate, sorbitan monooleate, sorbitan sesquioleate, sorbitan trioleate, and medium-chain triglyceride); glycerin fatty acid esters (e.g., glycerin fatty acid ester, glyceryl monostearate, glyceryl monomyristate, glyceryl monooleate, and glyceryl triisooctanoate); sucrose fatty acid esters (e.g., sucrose fatty acid ester, sucrose stearate, and sucrose palmitate); or propylene glycol fatty acid esters (e.g., propylene glycol fatty acid ester and propylene glycol monostearate).

9. The method according to claim 1 or the combination drug according to claim 2, wherein the lipophilic surfactant is sorbitan trioleate.

10. The method according to claim 1 or the combination drug according to claim 2, wherein the pharmaceutical composition further comprises an antioxidant agent B selected from the group consisting of tocopherols (e.g., α-tocopherol, β-tocopherol, γ-tocopherol, and δ-tocopherol); tocopherol acetate; and butylhydroxyanisole.

11. The method according to claim 1 or the combination drug according to claim 2, wherein the pharmaceutical composition further comprises an antioxidant B of α-tocopherol.

12. The method according to claim 1 or the combination drug according to claim 2, wherein the antioxidant agent A is ascorbyl palmitate, potassium ascorbate, sodium ascorbate, or ascorbic acid.

13. The method according to claim 1 or the combination drug according to claim 2, wherein the antioxidant agent A is sodium ascorbate or potassium ascorbate.

14. The method according to claim 1 or the combination drug according to claim 2, wherein the excipient is non-reducing sugars (e.g., sucrose and trehalose) or sugar alcohols (e.g., sorbitol, erythritol, xylitol, maltitol, and lactitol).

15. The method according to claim 1 or the combination drug according to claim 2, wherein the excipient is sucrose or trehalose.

16. The method according to claim 1 or the combination drug according to claim 2, wherein the increased amount in the area percentage value of an impurity UK-1.02 after a lyophilized formulation of the pharmaceutical composition is stored for 6 months at 5° C. is 5.0% or below.


SEQUENCE LISTING (2 sequences)

SEQ ID NO: 1 - protein, 181 aa, Artificial Sequence
  Pfs230D1+
DVGVDELDKIDLSYETTESGDTAVSEDSYDKYASQNTNKEYVCDFTDQLKPTESGPKVKKCEVKVNEPLIKVKIICPLKGSVEKLYDNIEYVPKKSPYVVLTKEETKLKEKLLSKLIYGLLISPTVNEKENNFKEGVIEFTLPPVVHKATVFYFICDNSKTEDDNKKGNRGIVEVYVEPYG

SEQ ID NO: 2 - protein, 187 aa, Artificial Sequence
  Pfs230D1+
DVGVDELDKIDLSYETTESGDTAVSEDSYDKYASQNTNKEYVCDFTDQLKPTESGPKVKKCEVKVNEPLIKVKIICPLKGSVEKLYDNIEYVPKKSPYVVLTKEETKLKEKLLSKLIYGLLISPTVNEKENNFKEGVIEFTLPPVVHKATVFYFICDNSKTEDDNKKGNRGIVEVYVEPYGHHHHHH